(19) United States
(12) Patent Application Publication
(10) Pub. No.: US 2025/0201408 A1
(43) Pub. Date: Jun. 19, 2025

(54) PREDICTION OF REPRESENTATIONS OF AN EXAMINATION AREA OF AN EXAMINATION OBJECT AFTER APPLICATIONS OF DIFFERENT AMOUNTS OF A CONTRAST AGENT

(71) Applicant: BAYER AKTIENGESELLSCHAFT, Leverkusen (DE)

(72) Inventors: Matthias LENGA, Leverkusen (DE); Gunnar SCHÜTZ, Leverkusen (DE); Stephan VÖLKENING, Monheim am Rhein (DE)

(21) Appl. No.: 18/840,596

(22) PCT Filed: Feb. 10, 2023
(86) PCT No.: PCT/EP2023/053324

§ 371 Date: Aug. 22, 2024

(30) Foreign Application Priority Data
Feb. 24, 2022 (EP) 22158528.4

(51) Int. Cl. G16H 50/20; A61B 5/00; A61B 5/055; A61B 6/00; A61B 6/03; A61B 8/00; A61B 8/08; G06V 10/25; G06V 10/774; G06V 10/776; G06V 20/50

(52) U.S. Cl. CPC G16H 50/20; A61B 5/0035; A61B 5/055; A61B 5/4244; A61B 5/7267; A61B 6/03; A61B 6/5247; A61B 8/08; A61B 8/481; A61B 8/5215; G06V 10/25; G06V 10/774; G06V 10/776; G06V 20/50; G06V 2201/031

(57) ABSTRACT

The present invention relates to the technical field of radiology, and in particular to assisting radiologists in radiological examinations using artificial intelligence methods. The present invention relates to training a machine learning model and using the trained model to predict representations of an examination area after applications of different amounts of a contrast agent.

The present invention relates to the technical field of radiology, in particular that of assisting radiologists in radiological examinations using artificial intelligence methods. The present invention is concerned with the training of a machine-learning model and the utilization of the trained model for predicting representations of an examination region after administration of different amounts of a contrast agent.

WO2019/074938A1 discloses a method for reducing the amount of contrast agent in the generation of radiological images with the aid of an artificial neural network.

In a first step, a training data set is generated. The training data set comprises for each person of a multiplicity of persons i) a native radiological image (zero-contrast image), ii) a radiological image after administration of a small amount of contrast agent (low-contrast image) and iii) a radiological image after administration of a standard amount of contrast agent (full-contrast image).

In a second step, an artificial neural network is trained to predict for each person of the training data set, on the basis of the native image and the image after administration of a low amount of contrast agent, an artificial radiological image showing an acquisition region after administration of the standard amount of contrast agent. The measured radiological image after administration of a standard amount of contrast agent is used in each case as reference (ground truth) in the training.

In a third step, the trained artificial neural network can be used to generate for a new person, on the basis of a native image and of a radiological image after administration of a small amount of contrast agent, an artificial radiological image showing the acquired region as it would look if a standard amount of contrast agent had been administered.

In the method disclosed in WO2019/074938A1, an artificial neural network is trained to map the native radiological image and the radiological image after administration of a small amount of contrast agent to the radiological image after administration of a standard amount of contrast agent. However, the artificial neural network is not trained to learn an increasing influence of contrast agent on a radiological image and thus cannot be used to generate radiological images showing an examination region after administration of different amounts of contrast agent.

In particular, the artificial neural network described in WO2019/074938A1 is unsuitable for generating radiological images showing an examination region after administration of an amount of contrast agent greater than the standard amount thereof.

Furthermore, the method described in WO2019/074938A1 for generating a radiological image showing an examination region after administration of the standard amount of a contrast agent always requires at least two radiological images (a native radiological image and a radiological image after administration of a small amount of the contrast agent).

Proceeding from the described prior art, it is an object of the invention to provide a machine-learning model, by means of which artificial radiological images showing an examination region after administration of different amounts of a contrast agent can be generated. Furthermore, it is intended that the model be capable in principle of generating artificial radiological images after administration of different amounts of contrast agent on the basis of a single image generated by measurement.

These objects are achieved by the subjects of the independent claims. Preferred embodiments can be found in the dependent claims, the present description and the drawings.

The present invention provides in a first aspect a computer-implemented method for training a machine-learning model. The training method comprises:
receiving training data,
   where the training data comprise representations ^{T}R₁ to ^{T}Rₙ of an examination region for a multiplicity of examination objects, where n is an integer greater than two,
   where each representation ^{T}Rᵢ represents the examination region after administration of an amount aᵢ of a contrast agent, where i is an integer in the range from 1 to n, where the amounts a₁ to aₙ form a sequence of preferably increasing amounts,
training the machine-learning model,
   where the model is trained to generate, starting from the representation ^{T}R₁, representations ^{T}R₂* to ^{T}Rₙ* one after the other,
   where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n, where the representation ^{T}Rₖ* represents the examination region after administration of the amount aₖ of the contrast agent and the representation ^{T}Rₖ₋₁* represents the examination region after administration of the amount aₖ₋₁ of the contrast agent,
   where differences between the generated representations ^{T}Rⱼ* and the representations ^{T}Rⱼ are quantified by means of a loss function, where j is an index passing through the numbers from 2 to n,
   where the differences are minimized in an optimization method by modifying model parameters of the machine-learning model,
storing the trained machine-learning model and/or using the machine-learning model for prediction.

The present invention further provides a computer-implemented method for predicting a representation of an examination region of an examination object. The prediction method comprises:
receiving a representation Rₚ of the examination region,
   where the representation Rₚ represents the examination region without contrast agent or after administration of an amount aₚ of a contrast agent, where p is an integer less than n, where n is an integer greater than 2,
feeding the representation Rₚ to a trained machine-learning model,
   where the machine-learning model has been trained on the basis of training data to generate, starting from a first representation ^{T}R₁, a number n−1 of representations ^{T}R₂* to ^{T}Rₙ* one after the other,
   where the first representation ^{T}R₁ represents the examination region without contrast agent or after administration of a first amount a₁ of the contrast agent and each generated representation ^{T}Rⱼ* represents the examination region after administration of an amount aⱼ of the contrast agent, where j is an index passing through integers from 2 to n, where the amounts a₁ to aₙ form a sequence of preferably increasing amounts,
   where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n,
receiving one or more representations R_{p+q}* of the examination region from the machine-learning model,
   where each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1,
outputting and/or storing and/or transmitting the one or more representations R_{p+q}*.

The present invention further provides a computer system comprising
an input unit,
a control and calculation unit and
an output unit,

wherein the control and calculation unit is configured
to cause the input unit to receive a representation Rₚ of an examination region of an examination object, where the representation Rₚ represents the examination region without contrast agent or after administration of an amount aₚ of a contrast agent, where p is an integer less than n, where n is an integer greater than 2,
to feed the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region without contrast agent or after administration of a first amount a₁ of the contrast agent and each generated representation ^{T}Rⱼ* represents the examination region after administration of an amount aⱼ of the contrast agent, where j is an index passing through integers from 2 to n, where the amounts a₁ to aₙ form a sequence of preferably increasing amounts, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n,
to receive from the machine-learning model one or more representations R_{p+q}* of the examination region, where each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1,
to cause the output unit to store the one or more representations R_{p+q}* and/or to output them and/or to transmit them to a separate computer system.

The present invention further provides a computer program product comprising a data memory in which there is stored a computer program that can be loaded into a working memory of a computer system, where it causes the computer system to execute the following steps:
receiving a representation Rₚ of an examination region of an examination object, where the representation Rₚ represents the examination region without contrast agent or after administration of an amount aₚ of a contrast agent, where p is an integer less than n, where n is an integer greater than 2,
feeding the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region without contrast agent or after administration of a first amount a₁ of the contrast agent and each generated representation ^{T}Rⱼ* represents the examination region after administration of an amount aⱼ of the contrast agent, where j is an index passing through integers from 2 to n, where the amounts a₁ to aₙ form a sequence of preferably increasing amounts, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n,
receiving one or more representations R_{p+q}* of the examination region from the machine-learning model, where each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1,
outputting and/or storing and/or transmitting the one or more representations R_{p+q}*.

The present invention further provides for a use of a contrast agent in a radiological examination method, wherein the radiological examination method comprises the following steps:
receiving a representation Rₚ of an examination region of an examination object, where the representation Rₚ represents the examination region without contrast agent or after administration of an amount aₚ of the contrast agent, where p is an integer less than n, where n is an integer greater than 2,
feeding the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region without contrast agent or after administration of a first amount a₁ of the contrast agent and each generated representation ^{T}Rⱼ* represents the examination region after administration of an amount aⱼ of the contrast agent, where j is an index passing through integers from 2 to n, where the amounts a₁ to aₙ form a sequence of preferably increasing amounts, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n,
receiving one or more representations R_{p+q}* of the examination region from the machine-learning model, where each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1,
outputting and/or storing and/or transmitting the one or more representations R_{p+q}*.

The present invention further provides a contrast agent for use in a radiological examination method, wherein the radiological examination method comprises the following steps:
generating a representation Rₚ of an examination region of an examination object, where the representation Rₚ represents the examination region without contrast agent or after administration of an amount aₚ of the contrast agent, where p is an integer less than n, where n is an integer greater than 2,
feeding the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region without contrast agent or after administration of a first amount a₁ of the contrast agent and each generated representation ^{T}Rⱼ* represents the examination region after administration of an amount aⱼ of the contrast agent, where j is an index passing through integers from 2 to n, where the amounts a₁ to aₙ form a sequence of preferably increasing amounts, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n,
receiving one or more representations R_{p+q}* of the examination region from the machine-learning model, where each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1,
outputting and/or storing and/or transmitting the one or more representations R_{p+q}*.

The present invention further provides a kit comprising a contrast agent and a computer program product, wherein the computer program product comprises a computer program that can be loaded into a working memory of a computer system, where it causes the computer system to execute the following steps:
receiving a representation Rₚ of an examination region of an examination object, where the representation Rₚ represents the examination region without contrast agent or after administration of an amount aₚ of the contrast agent, where p is an integer less than n, where n is an integer greater than 2,
feeding the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region without contrast agent or after administration of a first amount a₁ of the contrast agent and each generated representation ^{T}Rⱼ* represents the examination region after administration of an amount aⱼ of the contrast agent, where j is an index passing through integers from 2 to n, where the amounts a₁ to aₙ form a sequence of preferably increasing amounts, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n,
receiving one or more representations R_{p+q}* of the examination region from the machine-learning model, where each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1,
outputting and/or storing and/or transmitting the one or more representations R_{p+q}*.

The invention will be more particularly elucidated below without distinguishing between the subjects of the invention (training method, prediction method, computer system, computer program product, use, contrast agent for use, kit). Rather, the elucidations that follow are intended to apply analogously to all subjects of the invention, irrespective of the context (training method, prediction method, computer system, computer program product, use, contrast agent for use, kit) in which they occur.

Where steps are stated in an order in the present description or in the claims, this does not necessarily mean that the invention is limited to the order stated. Instead, it is conceivable that the steps are also executed in a different order or else in parallel with one another, the exception being when one step builds on another step, thereby making it imperative that the step building on the previous step be executed next (which will however become clear in the individual case). The orders stated are thus preferred embodiments of the invention.

With the aid of the present invention, representations of an examination region of an examination object can be predicted.

The “examination object” is normally a living being, preferably a mammal, most preferably a human.

The “examination region” is part of the examination object, for example an organ or part of an organ, such as the liver, brain, heart, kidney, lung, stomach, intestines or bladder or part of the aforementioned organs, or multiple organs or another part of the body.

In a preferred embodiment of the present invention, the examination region is the liver or part of the liver of a human.

The examination region, also referred to as the field of view (FOV), is in particular a volume that is imaged in radiological images. The examination region is typically defined by a radiologist, for example on a localizer image. It is of course also possible for the examination region to be alternatively or additionally defined in an automated manner, for example on the basis of a selected protocol.

A “representation of the examination region” is preferably a medical image. A “representation of the examination region” is preferably the result of a radiological examination.

“Radiology” is the branch of medicine that is concerned with the use of predominantly electromagnetic rays and mechanical waves (including for instance ultrasound diagnostics) for diagnostic, therapeutic and/or scientific purposes. Besides X-rays, other ionizing radiation such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present invention thus encompasses in particular the following examination methods: computed tomography, magnetic resonance imaging, sonography.

In a preferred embodiment of the present invention, the radiological examination is a computed tomography examination or a magnetic resonance imaging examination.

Computed tomography (CT) is an X-ray method which depicts the human body in cross-sectional images (sectional imaging method). Compared to a conventional X-ray image, on which usually only coarse structures and bones are identifiable, CT images also capture in detail soft tissues with small differences in contrast. An X-ray tube generates a so-called X-ray fan beam, which penetrates the body and is attenuated to varying degrees within the body owing to the various structures, such as organs and bones. The receiving detectors opposite the X-ray emitter receive the signals of varying strength and forward them to a computer, which puts together cross-sectional images of the body from the received data. Computed tomography images (CT images) can be observed in 2D or else in 3D. For better differentiability of structures within the body of the human (e.g., vessels), a contrast agent can be injected into, for example, a vein before CT images are generated.

Magnetic resonance imaging, MRI for short, is an imaging method that is used especially in medical diagnostics for depicting structure and function of tissues and organs in the human or animal body.

In MRI, the magnetic moments of protons in an examination object are aligned in a basic magnetic field, with the result that there is a macroscopic magnetization along a longitudinal direction. This is then deflected from the resting position by irradiation with high-frequency (HF) pulses (excitation). The return of the excited states to the resting position (relaxation), or magnetization dynamics, is then detected as relaxation signals by means of one or more HF receiver coils.

For spatial encoding, rapidly switched magnetic gradient fields are superimposed on the basic magnetic field. The captured relaxation signals, or detected MRI data, are initially present as raw data in frequency space, and can be transformed by subsequent inverse Fourier transform into real space (image space). Contrast agents can be used in magnetic resonance imaging, too, to achieve contrast enhancement.

“Contrast agents” are substances or mixtures of substances that improve the depiction of structures and functions of the body in radiological imaging methods.

Examples of contrast agents can be found in the literature (see for example A. S. L. Jascinth et al.: Contrast Agents in computed tomography: A Review, Journal of Applied Dental and Medical Sciences, 2016, vol. 2, issue 2, 143-149; H. Lusic et al.: X-ray-Computed Tomography Contrast Agents, Chem. Rev. 2013, 113, 3, 1641-1666; https://www.radiology.wisc.edu/wp-content/uploads/2017/10/contrast-agents-tutorial.pdf, M. R. Nouh et al.: Radiographic and magnetic resonances contrast agents: Essentials and tipsfor safe practices, World J Radiol. 2017 Sep. 28; 9(9): 339-349; L. C. Abonyi et al.: Intravascular Contrast Media in Radiography: Historical Development & Review of Risk Factors for Adverse Reactions, South American Journal of Clinical Research, 2016, vol. 3, issue 1, 1-10; ACR Manual on Contrast Media, 2020, ISBN: 978-1-55903-012-0; A. Ignee et al.: Ultrasound contrast agents, Endosc Ultrasound. 2016 November-December; 5(6): 355-362).

A representation of the examination region in the context of the present invention can be a computed tomogram, an MRI image, an ultrasound image or the like.

A representation of the examination region in the context of the present invention can be a representation in real space (image space), a representation in frequency space or some other representation. Preferably, representations of the examination region are present in a real-space depiction or in a form that can be converted (transformed) into a real-space depiction. A representation in real space is often also referred to as a pictorial depiction or as an image.

In a representation in real space, also referred to in this description as real-space depiction or real-space representation, the examination region is normally represented by a large number of image elements (pixels or voxels) that may for example be in a raster arrangement, in which case each image element represents a part of the examination region and each image element may be assigned a color value or gray value. A format widely used in radiology for storing and processing representations in real space is the DICOM format. DICOM (Digital Imaging and Communications in Medicine) is an open standard for storing and exchanging information in medical image data management.

A representation in real space can for example be converted (transformed) by a Fourier transform into a representation in frequency space. Conversely, a representation in frequency space can for example be converted (transformed) by an inverse Fourier transform into a representation in real space.

In a representation in frequency space, also referred to in this description as frequency-space depiction or frequency-space representation, the examination region is represented by a superposition of fundamental frequencies. For example, the examination region may be represented by a sum of sine and/or cosine functions having different amplitudes, frequencies and phases. The amplitudes and phases may be plotted as a function of the frequencies, for example, in a two- or three-dimensional representation. Normally, the lowest frequency (origin) is placed in the center. The further away from this center, the higher the frequencies. Each frequency can be assigned an amplitude representing the frequency in the frequency-space depiction and a phase indicating the extent of the shift of the respective wave with respect to a sine or cosine wave.

Details about real-space depictions and frequency-space depictions and their respective interconversion are described in numerous publications, see for example https://see.stanford.edu/materials/lsoftaee261/book-fall-07.pdf.

A representation in the context of the present invention represents the examination region before or after administration of an amount of a contrast agent.

The term “amount” may be understood to mean the absolute amount administered to an examination object (e.g., measured in kilograms or moles or liters); alternatively, the “amount” may be an administered dose, for example an amount of contrast agent (e.g., measured in kilograms or moles or liters) per kilogram body weight of the examination object. Alternatively, the term “amount” may be a concentration occurring in the examination object at least for a defined time after administration of the contrast agent. Other ways of defining amount are conceivable.

Preferably, the contrast agent is an MRI contrast agent. MRI contrast agents exert their effect by altering the relaxation times of structures that take up contrast agents. A distinction can be made between two groups of substances: paramagnetic and superparamagnetic substances. Both groups of substances have unpaired electrons that induce a magnetic field around the individual atoms or molecules.

Superparamagnetic contrast agents result in a predominant shortening of T2, whereas paramagnetic contrast agents mainly result in a shortening of T1. The effect of said contrast agents is indirect, since the contrast agent does not itself emit a signal, but instead merely influences the intensity of signals in its vicinity. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem®, Dotagita®, Cyclolux®), gadodiamide (Omniscan®), gadoteridol (ProHance®), and gadobutrol (Gadovist®).

Preferably, the MRI contrast agent is a hepatobiliary contrast agent. A hepatobiliary contrast agent has the characteristic features of being specifically taken up by liver cells (hepatocytes), accumulating in the functional tissue (parenchyma) and enhancing contrast in healthy liver tissue. An example of a hepatobiliary contrast agent is the disodium salt of gadoxetic acid (Gd-EOB-DTPA disodium), which is described in U.S. Pat. No. 6,039,931A and is commercially available under the trade names Primovist® and Eovist®.

The contrast agent may be administered in the form of a bolus injection, for example into an arm vein, in a dose based on body weight.

With the aid of the present invention, representations of an examination region after administration of different amounts of a contrast agent can be predicted. In principle, the prediction can be done on the basis of a single representation representing the examination region without contrast agent or after administration of a first amount of a contrast agent.

The prediction is done with the aid of a machine-learning model.

A “machine learning model” can be understood as meaning a computer-implemented data processing architecture. The model can receive input data and supply output data on the basis of said input data and model parameters. The model can learn a relationship between the input data and the output data by means of training. During training, model parameters can be adjusted so as to supply a desired output for a particular input.

During the training of such a model, the model is presented with training data from which it can learn. The trained machine learning model is the result of the training process. Besides input data, the training data include the correct output data (target data) that are to be generated by the model on the basis of the input data. During training, patterns that map the input data onto the target data are recognized.

In the training process, the input data of the training data are input into the model, and the model generates output data. The output data are compared with the target data. Model parameters are altered so as to reduce the differences between the output data and the target data to a (defined) minimum.

The differences can be quantified with the aid of a loss function. Such a loss function can be used to calculate a loss value for a given pair of output data and target data. The aim of the training process can consist in altering (adapting) the parameters of the machine learning model so that the loss value for all pairs of the training data set is reduced to a (defined) minimum.

For example, if the output data and the target data are numbers, the loss function can be the absolute difference between these numbers. In this case, a high absolute loss value can mean that one or more model parameters need to be altered to a substantial degree.

In the case of output data in the form of vectors, for example, difference metrics between vectors such as the mean squared error, a cosine distance, a norm of the difference vector such as a Euclidean distance, a Chebyshev distance, an Lp norm of a difference vector, a weighted norm or any other type of difference metric of two vectors can be chosen as the loss function.

In the case of higher-dimensional outputs, such as two-dimensional, three-dimensional or higher-dimensional outputs, an element-by-element difference metric can for example be used. Alternatively or in addition, the output data may be transformed into for example a one-dimensional vector before calculation of a loss value.

In the present case, the machine-learning model is trained on the basis of training data to predict, starting from a first representation representing an examination region without contrast agent or after administration of a first amount of a contrast agent, a sequence of representations representing the examination region after administration of different amounts of the contrast agent.

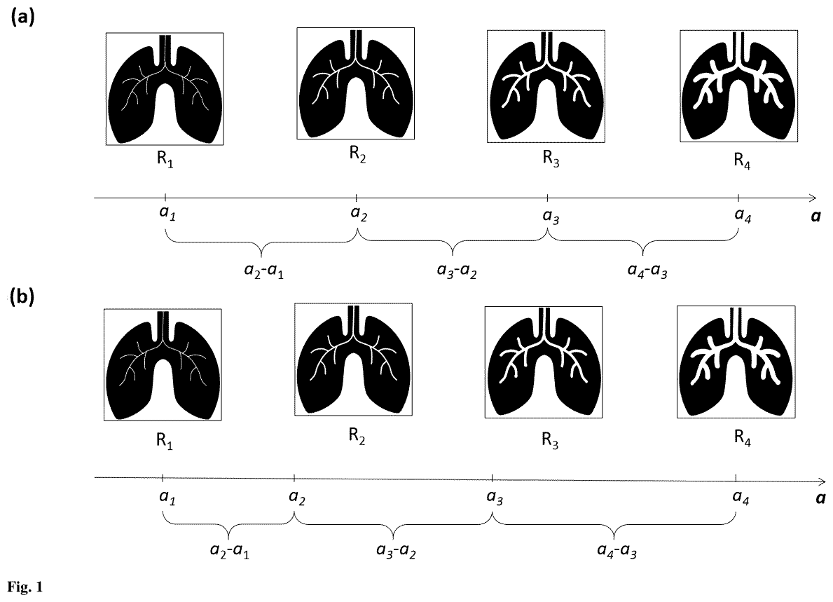

The different amounts of contrast agent form a sequence in which the amounts of contrast agent may, for example, increase or decrease. This shall be explained with reference to FIG. 1.

FIG. 1 shows, by way of example and in schematic form, four real-space representations of an examination region after administration of different amounts of a contrast agent. The examination region is the lung of a human. The four representations ^{T}R₁, ^{T}R₂, ^{T}R₃ and ^{T}R₄ are lined up along an axis indicating the amount a of concentration agent that has been administered in each case to the examination object. The representation ^{T}R₁ thus shows the examination region after administration of a first amount a₁ of the concentration agent; the representation ^{T}R₂ shows the examination region after administration of a second amount a₂ of the concentration agent; the representation ^{T}R₃ shows the examination region after administration of a third amount a₃ of the concentration agent; the representation ^{T}R₄ shows the examination region after administration of a fourth amount a₄ of the concentration agent. The first amount a₁ may be greater than or equal to zero, and the second amount a₂, the third amount a₃ and the fourth amount a₄ in the present example are each greater than zero. In the present example, the second amount a₂ is greater than the first amount a₁, the third amount a₃ is greater than the second amount a₂ and the fourth amount a₄ is greater than the third amount a₃: 0≤a₁<a₂<a₃<a₄

In the example shown in FIG. 1, the amounts of contrast agents thus form a sequence of increasing amounts of contrast agent.

This is shown in FIG. 1 in the representations ^{T}R₁ to ^{T}R₄ by an increasing contrast enhancement in the blood vessels compared to the surrounding tissue.

In the example shown in FIG. 1 (a), the amount increases by a constant magnitude, i.e., the difference between the amounts of two immediately consecutive amounts is the same for all immediately consecutive amounts: a₂−a₁=a₃−a₂=a₄−a₃

However, in a sequence of amounts of contrast agent, the magnitude by which the amounts increase may also vary. In the example shown in FIG. 1 (b), the amount increases by an increasing magnitude: a₂−a₁<a₃−a₂<a₄−a₃

It is alternatively possible that the amounts increase by a decreasing magnitude: a₂−a₁>a₃−a₂>a₄−a₃

It is alternatively possible that the representations have some other distribution along the amount axis.

Furthermore, it is possible that the amounts of contrast agent form a sequence of decreasing amounts or form some other sequence.

It should be noted that the different amounts of contrast agent are normally not administered in an immediately consecutive manner. An additive effect of the different amounts is normally to be prevented. In order to generate, for the training data, a data set for an examination object that represents two or more representations of an examination region after administration of different amounts of a contrast agent, the different amounts are normally administered at a time interval between each other that is sufficiently large for the amount of contrast agent from a previous administration present in the examination region to be sufficiently low at the time of a subsequent administration of a different amount of contrast agent for it not to exert a measurable influence on the representation of the examination region. In other words, the expression “the representation represents the examination region after an amount of a contrast agent” preferably means “the representation represents the examination region with an amount of a contrast agent”.

It should be noted that representations of the examination region are identified by the letter R in this description. The letter R can be followed by an index, for example R₁, R₂, R₃ or Rᵢ. The index added as a suffix indicates what is represented by the respective representation. The representation R₁ represents the examination region of an examination object after administration of the amount a₁ of a contrast agent; the representation R₂ represents the examination region of an examination object after administration of the amount a₂ of a contrast agent; the representation R₃ represents the examination region of an examination object after administration of the amount a₃ of a contrast agent, and so on. In general, the representation Rᵢ represents the examination region of an examination object after administration of the amount aᵢ of a contrast agent, where i is an index passing through the integers from i to n, where n is an integer greater than two. The expression “an index i passes through the integers from a to b” means that i assumes the values from a to b one after the other, i.e., it first assumes the value a (i=a), then the value a+1 (i=a+1), and so on until i reaches the value b (i=b). The expression “each generated representation Rⱼ* represents the examination region after administration of an amount aⱼ of the contrast agent” means that the representation R₁* represents the examination region after administration of an amount a₁ of the contrast agent, the representation R₂* represents the examination region after administration of an amount a₂ of the contrast agent, and so on. Especially in the claims and the drawings, representations which are used for training and representations which are generated in training are identified by a T added as a prefix, for example in the case of ^{T}R₁, ^{T}R₂ and ^{T}Rₖ₋₁*. Especially in the claims and the drawings, representations generated by means of the machine-learning model are identified by a superscript asterisk *, as in the case of R₁*, R₂* and ^{T}Rₖ₋₁*. The labels described herein serve merely for clarification and serve in particular to avoid any objections relating to clarity in the patent grant procedure.

The machine-learning model can be trained on the basis of training data to predict, starting from the first representation ^{T}R₁, the representations ^{T}R₂, ^{T}R₃ and ^{T}R₄ one after the other. In other words, the machine-learning model can learn the sequence of amounts of contrast agent so as to then apply said sequence to new representations.

The training will be explained below on the basis of three representations first of all for the sake of simplicity. This is followed by an extension to any desired number n of representations, where n is an integer greater than two.

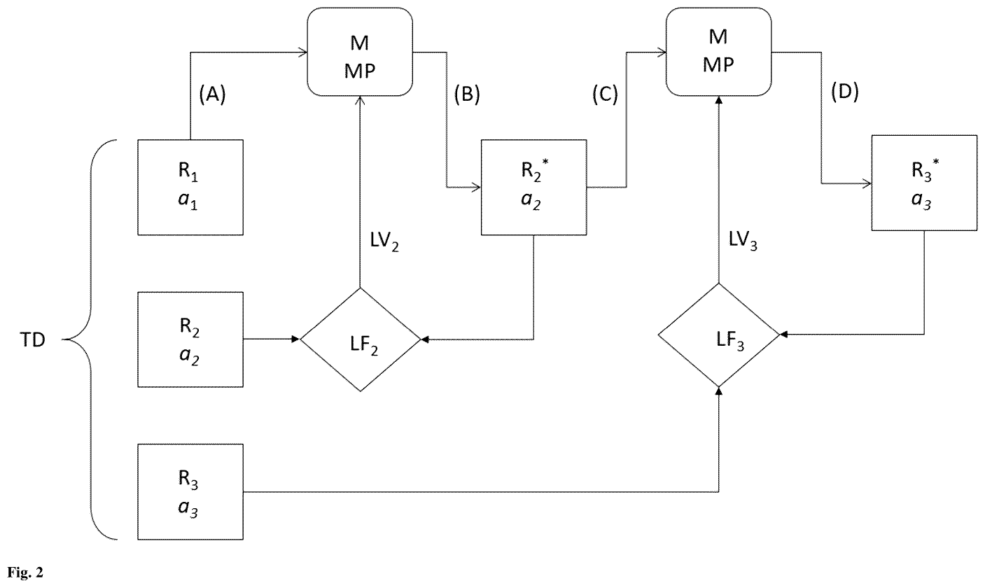

In the following explanation of the training on the basis of three representations, reference is made to FIG. 2.

FIG. 2 shows three representations: a first representation ^{T}R₁, a second representation ^{T}R₂ and a third representation ^{T}R₃.

The first representation ^{T}R₁ represents an examination region of an examination object without contrast agent or after administration of a first amount a₁ of a contrast agent, the second representation ^{T}R₂ represents the examination region after administration of a second amount a₂ of the contrast agent, and the third representation ^{T}R₃ represents the examination region after administration of a third amount a₃ of the contrast agent. The amounts of contrast agent a₁, a₂ and a₃ may form, for example, a sequence of increasing amounts of contrast agent (0≤a₁<a₂<a₃).

The three representations ^{T}R₁, ^{T}R₂ and ^{T}R₃ form a data set within training data TD. The training data TD comprise a multiplicity of such data sets. The term “multiplicity” preferably means more than 100. Each data set comprises at least two and usually three or more representations of the examination region after administration of the respective amounts of contrast agent (which may also be zero in the case of a₁).

The examination region is the same for all representations; only the examination object can vary; each data set usually comes from a different examination object. The statements made in this paragraph are generally applicable and do not apply just in relation to the example shown in FIG. 2.

In a first step (A), the first representation ^{T}R₁ is fed to a machine-learning model M. The machine-learning model M is configured to generate on the basis of the first representation ^{T}R₁ and on the basis of model parameters MP an output ^{T}R₂ (step (B)).

The output ^{T}R₂* is intended to approximate the second representation ^{T}R₂ as closely as possible; ideally, the output ^{T}R₂* cannot be distinguished from the second representation ^{T}R₂. In other words, the output ^{T}R₂* is a predicted second representation. The output ^{T}R₂* is compared with the (actual) second representation ^{T}R₂ and the differences are quantified with the aid of a loss function LF₂. In the case of multiple examination objects, a loss value LV₂ can be calculated for each pair of output ^{T}R₂* and second representation ^{T}R₂ by means of the loss function LF₂.

Examples of loss functions that can be used in general (not limited to the example in FIG. 2) to carry out the present invention are L1 loss function, L2 loss function, Lp loss function, structural similarity index measure (SSIM), VGG loss function, perceptual loss function or a combination of the aforementioned functions. Further details on loss functions can be found, for example, in the scientific literature (see for example: R. Mechrez et al.: The Contextual Loss for Image Transformation with Non-Aligned Data, 2018, arXiv: 1803.02077v4; H. Zhao et al.: Loss Functions for Image Restoration with Neural Networks, 2018, arXiv:1511.08861v3).

The output ^{T}R₂* is fed to the machine-learning model M in step (C). Even though FIG. 2 may suggest otherwise, the machine-learning model M to which the first representation ^{T}R₁ is fed in step (A) and that to which the generated representation ^{T}R₂* is fed in step (C) are the same model. This means that the machine-learning model M is not only configured to generate on the basis of the first representation ^{T}R₁ a representation ^{T}R₂*, but is also configured to generate from the representation ^{T}R₂* an output ^{T}R₃* approximating the third representation ^{T}R₃ as closely as possible. The output ^{T}R₃* is a predicted third representation. The output ^{T}R₃* is compared with the third representation ^{T}R₃. With the aid of a loss function LF₃, the difference between the output ^{T}R₃* and the third representation ^{T}R₃ can be quantified; in the case of multiple examination objects, a loss value LV₃ can be determined for each pair of a third representation ^{T}R₃ and a generated representation ^{T}R₃*.

Preferably, the machine-learning model undergoes end-to-end training. This means that the machine-learning model is simultaneously trained to generate a predicted second representation on the basis of the first representation and a predicted third representation on the basis of the predicted second representation. Preferably, this is achieved by using a loss function which takes into account both the differences between the predicted second representation and the second representation and the differences between the predicted third representation and the third representation.

It is possible to quantify the differences between the representation ^{T}R₂ and the generated representation ^{T}R₂* with the aid of the loss function LF₂ and to quantify the differences between the third representation ^{T}R₃ and the generated representation ^{T}R₃* with the aid of the loss function LF₃. A loss function LF taking into account both differences may for example be the sum of the individual loss functions: LF=LF₂+LF₃. It is also possible for the components formed by the individual loss functions LF_{z} and LF₃ in the total loss function LF to be weighted differently: LF=w₂·LF₂+w₃·LF₃, where w₂ and w₃ are weight factors that may assume, for example, values between 0 and 1. The value zero may be used for a weight factor, for example if a representation is missing in a data set (further details can be found later in the description). The weight factors may also be altered during training.

The training method shown in FIG. 2 is a preferred embodiment of the present disclosure and comprises the steps of:
receiving training data,
   where the training data comprise representations ^{T}R₁ to ^{T}R₃ of an examination region for a multiplicity of examination objects,
   where the representation ^{T}R₁ represents the examination region without contrast agent or after administration of an amount a₁ of a contrast agent, the representation ^{T}R₂ represents the examination region after administration of an amount a₂ of the contrast agent and the representation ^{T}R₃ represents the examination region after administration of an amount a₃ of the contrast agent, where the amounts a₁ to a₃ form a sequence of preferably increasing amounts,
training a machine-learning model, where the training for each examination object of the multiplicity of examination objects comprises:
   feeding the representation ^{T}R₁ to the machine-learning model, where the machine-learning model is configured to generate at least partly on the basis of the representation ^{T}R₁ and on the basis of model parameters a representation ^{T}R₂*,
   feeding the representation ^{T}R₂* to the machine-learning model, where the machine-learning model is further configured to generate at least partly on the basis of the representation ^{T}R₂* and on the basis of model parameters a representation ^{T}R₃*,
   quantifying differences between the representation ^{T}R₂ and the generated representation ^{T}R₂* and between the representation ^{T}R₃ and the generated representation ^{T}R₃*,
   minimizing the differences by modifying the model parameters,
storing the trained machine-learning model and/or using the machine-learning model for prediction.

When the trained machine-learning model is (later) used for prediction, this may be to predict a third representation on the basis of a first representation. For this purpose, it would be possible in principle to train a model to generate the third representation directly on the basis of the first representation (“direct training”). However, according to the invention, the machine-learning model is trained to generate the third representation on the basis of the first representation not in a direct manner (in one step), but in two steps, with a second representation being predicted in a first step, followed by prediction of the third representation on the basis of the second representation. The advantage of the iterative approach according to the invention over the aforementioned “direct training” is, inter alia, that additional training data (e.g., second representations representing the examination region in the second state) can be used, thus making it possible to achieve a higher accuracy of prediction. Furthermore, instead of learning how one representation is mapped onto another (or, as in the case of WO2019/074938A1, how multiple representations are mapped onto one representation), the model learns the influence exerted by contrast agent on the representations of the examination region, in particular how increasing or decreasing amounts of contrast agent have an effect on the representations. The greater the number of amounts of contrast agent covered by the training data, the more accurately the model can learn the influence of different amounts.

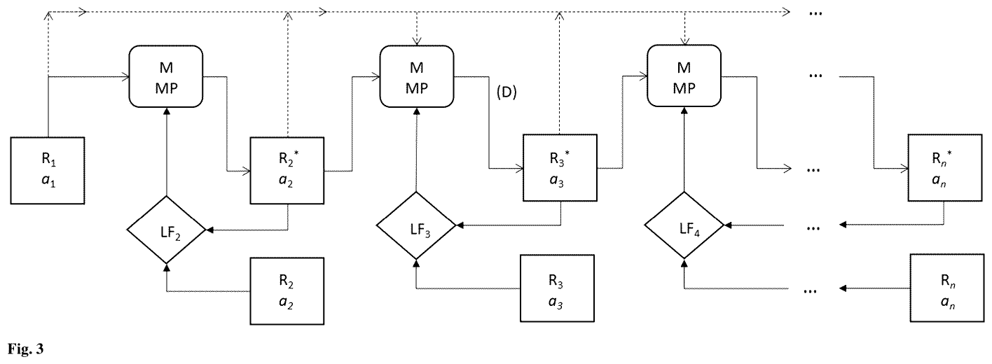

If the machine-learning model shown in FIG. 2 is trained, it can be used for prediction. This is shown in FIG. 3. In a first step (A), a first representation R₁ is fed to the trained machine-learning model M. The first representation R₁ represents the examination region of a new examination object after administration of the first amount a₁ of a contrast agent (which first amount a₁ may also be zero). The term “new” can mean that no representations of the examination object were used when training the machine-learning model M. The model M generates in step (B) on the basis of the first representation R₁ and on the basis of the model parameters MP a second representation R₂*. The second representation R₂* represents the examination region of the examination object after administration of the second amount a₂. In a third step (C), the generated second representation R₂* is fed to the machine-learning model M. The model M generates in step (D) on the basis of the second representation R₂* and on the basis of the model parameters MP a third representation R₃*. The third representation R₃* represents the examination region of the examination object after administration of the third amount a₃ of contrast agent.

The prediction method shown in FIG. 3 and based on the training method shown in FIG. 2 is a preferred embodiment of the present disclosure and comprises the steps of
receiving a representation R₁ of an examination region, where the representation R₁ represents the examination region without contrast agent or after administration of a first amount a₁ of a contrast agent,
generating a representation R₂* of the examination region with the aid of a trained machine-learning model at least partly on the basis of the representation R₁, where the generated representation R₂* represents the examination region after administration of a second amount a₂ of the contrast agent, where the second amount a₂ is preferably greater than zero and preferably greater than the first amount a₁,
generating a representation R₃* of the examination region with the aid of the machine-learning model at least partly on the basis of the generated representation R₂*, where the generated representation R₃* represents the examination region after administration of a third amount a₃ of the contrast agent, where the third amount a₃ is preferably greater than the second amount a₂,
outputting and/or storing and/or transmitting the representation R₃*,

where the machine-learning model has been trained on the basis of training data, where the training data comprise a multiplicity of data sets, where each data set comprises a first representation ^{T}R₁, a second representation ^{T}R₂ and a third representation ^{T}R₃, where the first representation ^{T}R₁ represents the examination region without contrast agent or after administration of the first amount a₁ of the contrast agent, the second representation ^{T}R₂ represents the examination region after administration of the second amount a₂ of the contrast agent and the third representation ^{T}R₃ represents the examination region after administration of the third amount a₃ of the contrast agent, where the machine-learning model has been trained to generate at least partly on the basis of the first representation ^{T}R₁ a representation ^{T}R₂* and to generate at least partly on the basis of the generated representation ^{T}R₂* a representation ^{T}R₃*, where the training involves quantifying differences between the representations ^{T}R₂ and ^{T}R₂* and between the representations ^{T}R₃ and ^{T}R₃* and modifying model parameters in order to minimize the differences.

In general, the machine-learning model can be trained to generate, starting from a first representation ^{T}R₁, a number n−1 of representations ^{T}R₂* to ^{T}Rₙ* one after the other, where n is an integer greater than 2, where the first representation ^{T}R₁ represents the examination region without contrast agent or after administration of a first amount a₁ of a contrast agent and each generated representation ^{T}Rⱼ* represents the examination region after administration of an amount aⱼ of the contrast agent, where j is an index passing through integers from 1 to n, where the amounts a₁ to aₙ form a sequence of preferably increasing amounts, where the representation ^{T}R₂* is generated at least partly on the basis of the representation ^{T}R₁ and each further representation ^{T}Rₖ* is generated at least partly on the basis of the respective previously generated representation ^{T}Rₖ₋₁*. where k is an index passing through integers from 3 to n.

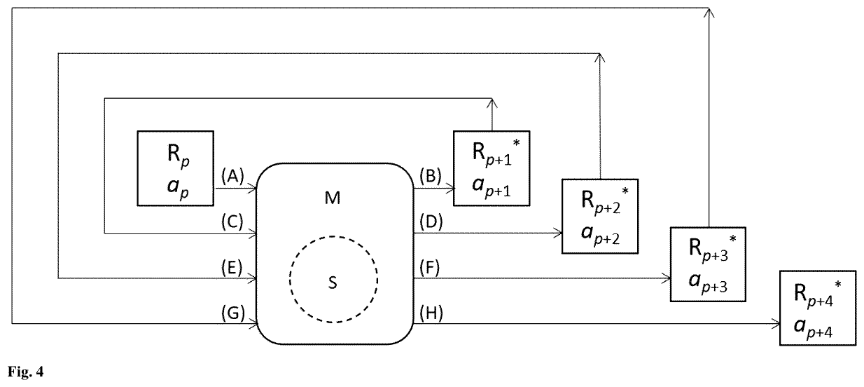

This shall be explained in greater detail using the example of FIG. 4. FIG. 4 can be understood as an extension of the scheme shown in FIG. 2 from three representations to any desired number (at least within defined limits) n of representations, where n is an integer greater than 2. Preferably, the number n is in the range from 3 to 100. However, the number n may also be greater than 100.

FIG. 4 shows a machine-learning model M. The machine-learning model M is specified three times, but it is always the same model. The machine-learning model M is trained to predict a number n−1 of representations one after the other. In the present example, n is an integer greater than 3. The model M is fed a first representation ^{T}R₁ as input data. The first representation ^{T}R₁ represents an examination region of an examination object without contrast agent or after administration of a first amount a₁ of a contrast agent. The machine-learning model M is configured to generate at least partly on the basis of the first representation ^{T}R₁ and on the basis of model parameters MP an output ^{T}R₂*. The output ^{T}R₂* is a predicted second representation representing the examination region after administration of a second amount a₂ of the contrast agent. The model is further configured to generate at least partly on the basis of the output ^{T}R₂* and model parameters MP an output ^{T}R₃*. The output ^{T}R₃* is a predicted third representation representing the examination region after a third amount a₃ of the contrast agent. The model is further configured to generate at least partly on the basis of the output ^{T}R₃* and model parameters MP a further output representing a predicted representation of the examination region after administration of a further amount of the contrast agent. This scheme is continued up to an output ^{T}Rₙ*. The output ^{T}Rₙ* is a predicted n-th representation representing the examination region after administration of an n-th amount an of the contrast agent. The amounts of contrast agent a₁, a₂, a₃, . . . aₙ form a sequence. The sequence may be, for example, a sequence of increasing amounts of contrast agent. In other words, it is preferred that the second amount a₂ is greater than zero and greater than the first amount a₁, that the third amount a₃ is greater than the second amount a₂, that the fourth amount a₄ is greater than the third amount a₃, and so on. In general terms, the j-th amount aⱼ is preferably greater than the (j−1)-th amount aⱼ₋₁, where j is an index passing through the numbers from 2 to n.

Present in the case of the example shown in FIG. 4 are not only the first representation ^{T}R₁, but also further (actual, measurement-generated) representations ^{T}Rⱼ (where j=2 to n) of the examination region after administration of the amounts aⱼ (where j=2 to n) of the contrast agent that can be used as ground truth data for training of the machine-learning model. For instance, the difference between an output ^{T}Rⱼ* (which is a predicted j-th representation of the examination region after administration of an amount aⱼ of the contrast agent) and the representation ^{T}Rⱼ may be quantified using a loss function LFⱼ: a loss function LF₂ may quantify the difference between the representation ^{T}R₂ and the generated representation ^{T}R₂*, a loss function LF₃ may quantify the difference between the representation ^{T}R₃ and the generated representation ^{T}R₃*, and so on.

In an end-to-end training, a total loss function LF taking into account all individual differences may be used; for example, this may be the sum of the individual differences:

$$LF = {LF}_{2} + {LF}_{3} + … + {LF}_{n} = {\sum }_{j = 2}^{n} {LF}_{j}$$

Alternatively, the sum may be a weighted sum:

$$LF = {w}_{2} \cdot {LF}_{2} + {w}_{3} \cdot {LF}_{3} + … + {w}_{n} \cdot {LF}_{n} = {\sum }_{j = 2}^{n} {w}_{j} \cdot {LF}_{j}$$

where the weight factors wᵢ may assume, for example, values from 0 to 1.

The advantage of weighting is that the representations fed to the machine-learning model are given differing weight when generating a predicted representation.

For example, it is possible that the weight factors wⱼ increase in the order w₂, w₃, . . . , wₙ with the amount of contrast agent a₂, a₃, . . . , aₙ and thus more weight is given to representations associated with an amount of contrast agent closer to the amount an. The increase may be logarithmic, linear, square, cubic or exponential, or it may be some other increase. It is also conceivable to give less weight to the representations having a greater amount of contrast agent, in that the weight factors decrease in the order w₂, w₃, . . . , wₙ with increasing amount of contrast agent a₂, a₃, . . . , aₙ and thus more weight is given to representations closer to the initial representation R₁. In the case too of such a decrease, it may be logarithmic, linear, square, cubic or exponential, or it may be some other decrease.

It is also conceivable that the training data comprise incomplete data sets. This means that some data sets of examination objects do not comprise all representations ^{T}R₁ to ^{T}Rₙ. As explained later in the description, incomplete data sets may also be used for training of the machine-learning model. If a representation ^{T}Rⱼ is missing, the weight factor wⱼ which is to weight the loss function relating to the representation ^{T}Rⱼ and the generated representation ^{T}Rⱼ* may be set to zero, where j is an integer which may assume the values of 2 to n.

It is also conceivable that training involves considering random numbers 1≤j<k≤n and the associated partial sequences of amounts of contrast agent aⱼ, aⱼ₊₁, . . . , aₖ₋₁, aₖ. The learning problem described above may then be solved on the basis of these partial sequences (which optionally vary from time to time). For example, it is conceivable that a random initial amount of contrast agent aⱼ (1≤j≤n−2) is determined and the model is to always synthesize the representations of the subsequent two amounts of contrast agent aⱼ₊₁, aⱼ₊₂ on the basis thereof.

In FIG. 4, it is also shown schematically that a representation ^{T}Rⱼ* representing the examination region after administration of an amount aⱼ of the contrast agent may be generated by using not only the representation ^{T}Rⱼ₋₁* representing the examination region after administration of an amount aⱼ₋₁, but also further representations ^{T}Rⱼ₋₂* and/or ^{T}Rⱼ₋₃* and/or . . . to ^{T}R₁ representing the examination region after administration of the further amounts aⱼ₋₂, aⱼ₋₃ . . . to a₁, where j is an integer which may assume the values of 2 to n. This is expressed by the dashed arrows. For instance, the representation ^{T}R₃* may be generated by feeding not only the representation ^{T}R₂*, but also the representation ^{T}R₁, to the machine-learning model. The representation ^{T}R₄* may be generated by feeding not only the representation ^{T}R₃*, but also the representation ^{T}R₂* and/or the representation ^{T}R₁, to the machine-learning model.

To predict representations, additional information may also be incorporated into the machine-learning model, such as information about the amount of contrast agent that has been administered in each case. In other words, the second representation may be predicted by using not only the first representation, but also information about the amount of administered contrast agent represented by the first representation. The predicted third representation, too, may be generated on the basis of the predicted second representation and information about the second amount of contrast agent. If information is provided about the amount of contrast agent associated with a representation, the machine-learning model will “know” where in the sequence of amounts of contrast agent it is in.

Examples of additional information that may be used for training of and later prediction by the machine-learning model are information about the examination region, information about the examination object, information about the conditions when the representation was generated, information about the contrast agent, information about the amount of contrast agent administered to the examination object, and other/additional information.

The machine-learning model of the present disclosure may be understood to mean a transformation which may be applied to a representation of an examination region in one state of a sequence of states in order to predict a representation of the examination region in a subsequent state of the sequence of states. Each state is represented by an amount of contrast agent administered. The transformation may be applied singly (once) in order to predict a representation of the examination region in the immediately subsequent state, or it may be applied multiply (multiple times) in order to predict a representation of the examination region in one state further downstream in the sequence of states.

If there is a sequence of n states Z₁ to Zₙ, and if at each state Zᵢ there is a representation Rᵢ representing the examination region in the state Zᵢ, the machine-learning model M may be applied q times starting from the representation R₁ in order to generate a representation R₁, representing the examination region in the state Z₁₄₉, where q may assume the values of 1 to n−1:

$$\begin{matrix}{M}^{q} ( {R}_{1} ) = {R}_{1 + q}^{*} \\ q = 1 \text{:} M ( {R}_{1} ) = {R}_{2}^{*} \\ q = 2 \text{:} M ( {R}_{2}^{*} ) = M ( M ( {R}_{1} ) ) = {M}^{2} ( {R}_{1} ) = {R}_{3}^{*} \\ q = 3 \text{:} M ( {R}_{3}^{*} ) = M ( M ( {R}_{2}^{*} ) ) = M ( M ( M ( {R}_{1} ) ) ) = {M}^{3} ( {R}_{1} ) = {R}_{4}^{*} \\ … \\ q = n - 1 \text{:} M ( {R}_{n - 1}^{*} ) = M ( M ( {R}_{n - 2}^{*} ) ) = … = {M}^{n - 1} ( {R}_{1} ) = {R}_{n}^{*}\end{matrix}$$

This also applies analogously to the training method, in which the q-times transformation M may be described by the following formula:

$${M}^{q} {(}^{T} {R}_{1} ) = {}^{T} {R}_{1 + q}^{*}$$

The respective state indicates the amount of contrast agent administered. A sequence of states may thus, for example, characterize the examination region after administration of a sequence of increasing amounts of contrast agent.

A loss function quantifying all differences between predicted representations ^{T}R_{q}* and actual (measurement-generated) representations ^{T}R_{q} may be expressed, for example, by the following formula:

$$LV = {w}_{2} \cdot d ( M ( {}^{T} {R}_{1} ) , {}^{T} {R}_{2} ) + {w}_{3} \cdot d ( {M}^{2} ( {}^{T} {R}_{1} ) , {}^{T} {R}_{3} ) + … + {w}_{n} \cdot d ( {M}^{n - 1} ( {}^{T} {R}_{1} ) , {}^{T} {R}_{n} )$$

Here, LV is the loss value produced for a data set comprising the (actual, measurement-generated) representations ^{T}R₁, ^{T}R₂, . . . , ^{T}Rₙ. d is a loss function quantifying the differences between a predicted representation M(^{T}R_{q-1}) and the representation ^{T}R_{q}. As already described earlier in the description, this may be, for example, one of the following loss functions: L1 loss, L2 loss, Lp loss, structural similarity index measure (SSIM), VGG loss, perceptual loss or a combination thereof.

w₂, w₃, . . . , wₙ are weight factors that have likewise already been described earlier in the description.

n indicates the number of states (the number of different amounts of contrast agent).

It is also conceivable that the loss value is the maximum difference calculated for a data set:

$$LV = max ( {w}_{2} \cdot d ( M ( {}^{T} {R}_{1} ) , {}^{T} {R}_{2} ) ; {w}_{3} \cdot d ( {M}^{2} ( {}^{T} {R}_{1} ) , {}^{T} {R}_{3} ) ; … ; {w}_{n} \cdot d ( {M}^{n - 1} ( {}^{T} {R}_{1} ) , {}^{T} {R}_{n} ) )$$

and in this formula too, the weight factors can also be used to give a different weight to the individual differences.

As already indicated earlier in the description, it should be noted that training of the machine-learning model does not require that each data set of the training data contain representations of all amounts of contrast agent to be learnt by the model. In principle, two representations of the examination region after administration of two different amounts of contrast agent are sufficient. This shall be explained using an example. Assume that the machine-learning model is to be trained to predict representations of an examination region with the increasing amounts of contrast agent a₁ to a₆. Assume that training data comprising 10 data sets of 10 examination objects are sufficient for training the machine-learning model.

Each data set comprises representations of the examination region after administration of different amounts of contrast agent, for example:
Data set 1: ^{T}R₁, ^{T}R₃, ^{T}R₄, ^{T}R₅, ^{T}R₆
Data set 2: ^{T}R₁, ^{T}R₂, ^{T}R₃, ^{T}R₆
Data set 3: ^{T}R₁, ^{T}R₂, ^{T}R₃, ^{T}R₄, ^{T}R₅
Data set 4: ^{T}R₁, ^{T}R₂, ^{T}R₃, ^{T}R₅, ^{T}R₆
Data set 5: ^{T}R₂, ^{T}R₃, ^{T}R₅, ^{T}R₆
Data set 6: ^{T}R₂, ^{T}R₃, ^{T}R₄, ^{T}R₅, ^{T}R₆
Data set 7: ^{T}R₂, ^{T}R₃, ^{T}R₅, ^{T}R₆
Data set 8: ^{T}R₃, ^{T}R₅, ^{T}R₆
Data set 9: ^{T}R₃, ^{T}R₄, ^{T}R₅, ^{T}R₆
Data set 10: ^{T}R₃, ^{T}R₄, ^{T}R₆

In the example, there is no single data set that is “complete”, i.e., no data set comprising all possible representations ^{T}R₁, ^{T}R₂, ^{T}R₃, ^{T}R₄, ^{T}R₅ and ^{T}R₆. Nevertheless, it is possible to train the machine-learning model on the basis of such training data to predict a representation of the examination region after administration of each amount a₂ to a₆. This is another advantage of the present invention over the “direct training” described above.

Once the machine-learning model is trained, the model can be fed new (i.e., not used in training) representations of an examination region after administration of an amount of the contrast agent, and the model can predict (generate) one or more representations of the examination region after administration of a different (greater) amount.

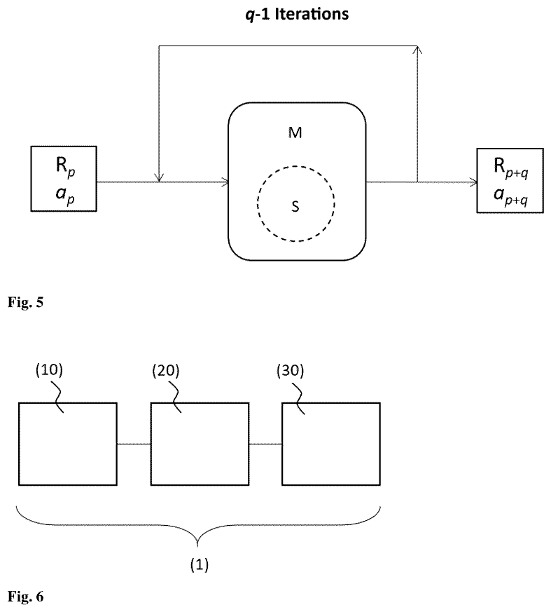

This is depicted by way of example and in schematic form in FIG. 5. In the example shown in FIG. 5, what is generated starting from a representation Rₚ representing the examination region after administration of the amount aₚ of a contrast agent is a sequence of representations Rₚ₊₁*, Rₚ₊₂*, Rₚ₊₃* and Rₚ₊₄* representing the examination region after administration of the amounts aₚ₊₁, aₚ₊₂, aₚ₊₃ and aₚ₊₄. The amounts of contrast agent aₚ₊₁, aₚ₊₂, aₚ₊₃ and aₚ₊₄ preferably form a sequence of increasing amounts: aₚ₊₁<aₚ₊₂<aₚ₊₃<aₚ₊₄.

In step (A), the machine-learning model M is fed the representation Rₚ. Besides the representation Rₚ, the machine-learning model M may also be fed information about the amount of contrast agent aₚ and/or additional/other information, as described earlier in this description.

The machine-learning model M generates in step (B) on the basis of the fed input data the representation Rₚ₊₁* representing the examination region after administration of the amount aₚ₊₁.

In step (C), the machine-learning model M is fed the previously generated representation Rₚ₊₁*. Besides the representation Rₚ₊₁*, the machine-learning model M may also be fed information about the amount of contrast agent aₚ₊₁ and/or additional information. Furthermore, the machine-learning model may also be fed the representation Rₚ and/or information about the amount of contrast agent aₚ.

Preferably, the machine-learning model M comprises a memory S which stores input data (and preferably also output data), so that one-time entered input data and/or generated output data need not be received and/or entered again, but are already available to the machine-learning model. This applies not only to the utilization of the trained machine-learning model for prediction as described in this example, but also to the training of the machine-learning model according to the invention and, in general as well, to all other embodiments.

The machine-learning model M generates in step (D) on the basis of the fed input data the representation Rₚ₊₂* representing the examination region after administration of the amount of contrast agent aₚ₊₂.

In step (E), the machine-learning model M is fed the previously generated representation Rₚ₊₂*. Besides the representation Rₚ₊₂*, the machine-learning model M may also be fed information about the amount of contrast agent aₚ₊₂ and/or additional information. Furthermore, the machine-learning model may also be fed the representation Rₚ and/or the representation Rₚ₊₁* and/or information about the amounts of contrast agent aₚ and/or aₚ₊₁.

The machine-learning model M generates in step (F) on the basis of the fed input data the representation Rₚ₊₃* representing the examination region after administration of the amount of contrast agent aₚ₊₃.

In step (G), the machine-learning model M is fed the previously generated representation Rₚ₊₃*. Besides the representation Rₚ₊₃*, the machine-learning model M may also be fed information about the amount of contrast agent aₚ₊₃ and/or additional information. Furthermore, the machine-learning model may also be fed the representation Rₚ and/or the representation Rₚ₊₁* and/or the representation Rₚ₊₂* and/or information about the amounts of contrast agent aₚ and/or aₚ₊₁ and/or aₚ₊₂.

The machine-learning model M generates in step (H) on the basis of the fed input data the representation Rₚ₊₄* representing the examination region after administration of the amount of contrast agent aₚ₊₄.

The generated representations Rₚ₊₁*, Rₚ₊₂*, Rₚ₊₃* and/or Rₚ₊₄* may be output (e.g., displayed on a monitor and/or printed by a printer) and/or stored in a data memory and/or transmitted to a (separate) computer system.

If the machine-learning model according to the invention has been trained to generate, starting from a first representation ^{T}R₁ representing the examination region after administration of a first amount a₁ of the contrast agent, a sequence of predicted representations ^{T}R₁*, ^{T}R₂*, . . . , ^{T}Rₙ*, where each predicted representation ^{T}Rⱼ* represents the examination region after administration of an amount aⱼ of the contrast agent and where j is an index passing through integers from 2 to n, then such a model can be fed a new representation Rₚ representing the examination region after administration of the amount aₚ of the contrast agent and the machine-learning model can generate the predicted representations Rₚ₊₁*, Rₚ₊₂*, . . . , Rₙ* one after the other, where p is a number which may assume the values of 2 to n.

This means that the trained model does not necessarily have to be fed a new representation R₁ representing the examination region after administration of the first amount a₁, and there is also no limiting of the trained machine-learning model to predicting only the representation Rₙ representing the examination region after administration of the (preferably greatest) amount aₙ. Instead, it is possible to “drop in” and “drop out” anywhere in the sequence of amounts of contrast agent a₁ to aₙ, i.e., it is possible to predict, on the basis of any desired representation in relation to the sequence of amounts of contrast agent, any other desired representation in relation to an amount of contrast agent that follows in the sequence of amounts of contrast agent.

Thus, if a machine-learning model has been trained, for example, to learn the effect of an increasing amount of contrast agent in a sequence of increasing amounts of contrast agent a₁, a₂, a₃, a₄, then the trained machine-learning model may for example
be fed a representation R₁ in order to generate a representation R₂* and/or R₃* and/or R₄*, or
be fed a representation R₂ in order to generate a representation R₃* and/or R₄*, or
be fed a representation R₃ in order to generate a representation R₄*,

where the representation R₁ represents the examination region after administration of the amount a₁, the representations R₂ and R₂* represent the examination region after administration of the amount a₂, the representations R₃ and R₃* represent the examination region after administration of the amount a₃, and the representations R₄ and R₄* represent the examination region after administration of the amount a₄.

It is even possible to generate representations representing the examination region after administration of an amount of contrast agent that was not addressed at all in the training. Thus, instead of stopping at the representation Rₙ* (e.g., R₄* in the example above), it is also possible to generate a representation Rₙ₊₁* (e.g., R₅*), Rₙ₊₂* (e.g., R₆) and so on. The trained machine-learning model may thus be used to continue the learned effect of a preferably increasing amount of contrast agent and to calculate representations that were never generated by measurement. In this respect, the trained machine-learning model may be used to extrapolate to very high amounts of contrast agent that would never be administered to an examination object.

Furthermore, the predictions are not limited to increasing amounts of contrast agent. It is also possible to predict representations with decreasing amounts of contrast agent. Firstly, the machine-learning model may fundamentally be trained in both directions: in the direction of increasing amounts of contrast agent and in the direction of decreasing amounts of contrast agent. Secondly, the machine-learning model performs, on a representation entered into the model, a transformation that in principle can also be reversed. Analysis of the learnt mathematical functions of the model that transform an input representation into an output representation makes it possible to determine inverse functions that reverse the process and change the previous output representation back into the previous input representation. The inverse functions may then be used to predict representations with decreasing amounts of contrast agent, even if the model was trained to predict representations with increasing amounts of contrast agent, and vice versa.

FIG. 6 shows an extension of the scheme shown in FIG. 5. Whereas FIG. 5 shows how, on the basis of a representation Rₚ representing the examination region after administration of an amount of contrast agent aₚ, the representations Rₚ₊₁*, Rₚ₊₂*, Rₚ₊₃* and Rₚ₊₄* representing the examination region after administration of the amounts aₚ₊₁, aₚ₊₂, aₚ₊₃ and aₚ₊₄ of the contrast agent are generated one after the other, FIG. 5 shows how, on the basis of the representation Rₚ representing the examination region after administration of the amount of contrast agent aₚ, the representations Rₚ₊₁* to Rₚ₊₄* are generated one after the other, where p is an integer which may assume the values of 2 to n (or else values greater than n, as described), and q is an integer greater than 1. In FIG. 6, the iterative character of the machine-learning model M is expressed particularly clearly. The machine-learning model M is, starting from the representation Rₚ, applied q times, and the output data are returned (q−1) times back into the machine-learning model.

It should be noted that the (trained or untrained) machine-learning model does not have to be applied to a complete radiological image (e.g., an MRI image or a CT scan or the like). It is possible to apply the machine-learning model to only part of a radiological image. It is possible, for example, to segment a radiological image first in order to identify/select a region of interest. The model may then, for example, be applied solely to the region of interest.

Furthermore, the application of the machine-learning model may comprise one or more preprocessing and/or postprocessing steps. For example, it is conceivable to first subject a received representation of the examination region to one or more transformations, such as motion correction, color space conversion, normalization, segmentation, Fourier transform (e.g., for conversion from an image-space representation into a frequency-space representation), inverse Fourier transform (e.g., for conversion from a frequency-space representation into an image-space representation) and/or the like. In a further step, the transformed representation may be fed to the machine-learning model, which then passes through a series of iterations (cycles) in order to generate, starting from the transformed representation, a series of further (subsequent) representations of the examination region in a series of further (subsequent) states.

The machine-learning model according to the invention may, for example, be an artificial neural network or include such a network.

An artificial neural network comprises at least three layers of processing elements: a first layer with input neurons (nodes), an N-th layer with at least one output neuron (nodes) and N−2 inner layers, where N is a natural number and greater than 2.

The input neurons serve to receive the representations. There is normally one input neuron for each pixel or voxel of a representation when the representation is a real-space depiction in the form of a raster graphic. There may be additional input neurons for additional input values (e.g., information about the examination region, information about the examination object, information about the conditions when the representation was generated, information about the contrast agent, information about the amount of contrast agent administered to the examination object, and other/additional information).

The output neurons can serve to output a predicted representation.

The processing elements of the layers between the input neurons and the output neurons are connected to one another in a predetermined pattern with predetermined connection weights.

Preferably, the artificial neural network is a so-called convolutional neural network (CNN for short) or includes such a network.

A CNN normally consists essentially of an alternately repeating array of filters (convolutional layer) and aggregation layers (pooling layer) terminating in one or more layers of fully connected neurons (dense/fully connected layer).

The training of the neural network can, for example, be carried out by means of a backpropagation method. The for the network is to predict the dynamics of the examination region from one state via at least one intermediate state to an end state as reliably as possible. The quality of prediction is described by a loss function. The goal is to minimize the loss function. In the case of the backpropagation method, an artificial neural network is taught by the alteration of the connection weights.

In the trained state, the connection weights between the processing elements contain information regarding the effect of an increasing or decreasing amount of contrast agent on the examination region that may be used in order to predict, on the basis of a first representation representing the examination region after administration of a first amount of the contrast agent, one or more representations representing the examination region after administration of a different amount of the contrast agent.

A cross-validation method may be used in order to divide the data into training and validation data sets. The training data set is used in the backpropagation training of network weights. The validation data set is used in order to check the accuracy of prediction with which the trained network can be applied to unknown data.

The artificial neural network may have an autoencoder architecture; for example, the artificial neural network may have an architecture such as U-Net (see for example O. Ronneberger et al.: U-net: Convolutional networks for biomedical image segmentation, International Conference on Medical image computing and computer-assisted intervention, pages 234-241, Springer, 2015, https://doi.org/10.1007/978-3-319-24574-4_28).

The artificial neural network may be a generative adversarial network (GAN) (see for example M.-Y. Liu et al.: Generative Adversarial Networks for Image and Video Synthesis: Algorithms and Applications, arXiv:2008.02793; J. Henry et al.: Pix2Pix GAN for Image-to-Image Translation, DOI: 10.13140/RG.2.2.32286.66887).

The artificial neural network may be a recurrent neural network or include such a network. Recurrent or feedback neural networks refer to neural networks that, in contrast to feed-forward networks, are distinguished by connections between neurons of one layer and neurons of the same layer or a preceding layer. The artificial neural network may for example include a long short-term memory (LSTM) (see for example Y. Gao et al.: Fully convolutional structured LSTM networks for joint 4D medical image segmentation, DOI: 10.1109/ISBI.2018.8363764).

The artificial neural network may be a transformer network (see for example D. Karimi et al.: Convolution-Free Medical Image Segmentation using Transformers, arXiv:2102.13645 [eess.IV]).

The present invention may be used, for example, to reduce the amount of contrast agent in a radiological examination.

For each contrast agent, there is a recommended amount that may be given to an examination object for a defined purpose. Contrast agents usually have authorization for a defined purpose, said authorization including information about the amount to be given (to be administered). An amount that is recommended by a manufacturer and/or distributor of a contrast agent or an amount of a contrast agent that is stipulated under an authorization is referred to as standard amount in this description.

For example, the standard amount of Gd-EOB-DTPA disodium is 0.025 mmol/kg body weight.

The present invention may be used, for example, to predict, on the basis of a representation which an examination region after administration of an amount of a contrast agent smaller the standard amount of the contrast agent, a representation of the examination region after administration of a standard amount.

To this end, a machine-learning model may trained on the basis of training data to learn the influence of an increasing amount of contrast agent on the representation of the examination region.

The training data set may include, for example, not only a representation representing the examination region after administration of the standard amount of the contrast agent, but also a plurality of representations representing the examination region after administration of different amounts of concentration agent, said amounts usually being smaller than the standard amount. For example, it is possible that the training data include, for each examination object of a multiplicity of examination objects, one or more of six representations: a first representation, a second representation, a third representation, a fourth representation, a fifth representation and a sixth representation.

The first representation may represent the examination region without contrast agent. The second representation may represent the examination region after administration of, for example, 20% of the standard amount of the contrast agent. The third representation may represent the examination region after administration of, for example, 40% of the standard amount of the contrast agent. The fourth representation may represent the examination region after administration of, for example, 60% of the standard amount of the contrast agent. The fifth representation may represent the examination region after administration of, for example, 80% of the standard amount of the contrast agent. The sixth representation may represent the examination region after administration of, for example, 100% of the standard amount of the contrast agent.

The machine-learning model may be trained on the basis of the training data to predict, starting from a representation which the examination region after administration of an amount of the contrast agent (which may also be zero), a different representation representing the examination region after administration of a different amount of the contrast agent by single or multiple application of the machine-learning model.

Once the model is trained, it can be used for prediction. To this end, the trained machine-learning model may be fed a representation of the examination region (of a different examination object not considered during the training) representing the examination region after administration of an amount of the contrast agent representing 0% or 20% or 40% or 60% or 80% of the standard amount of the contrast agent, and by application of the trained machine-learning model five times (in the case of 0%), four times (in the case of 20%), three times (in the case of 40%), two times (in the case of 60%) or once (in the case of 80%), the model can be used to predict a representation of the examination region representing the examination region after administration of the standard amount of the contrast agent.

In another application example, the present invention may be used to predict a representation representing the examination region after administration of an amount greater than the standard amount, without having to administer such a large amount to an examination object during the training.

To stay with the abovementioned example, the trained machine-learning model may be applied repeatedly to predict a representation of the examination region representing the examination region after administration of 120% or 140% or 160% or 180% or 200% of the standard amount. Values beyond 200% are also possible. Even if it would not be possible to verify that a predicted representation would in fact have such an appearance in an actual case, since administration of such large amounts would not be justifiable for ethical reasons, such a predicted representation may be of benefit if dysfunctions and/or lesions and/or the like are identified in such images by a radiologist who would not identify them or would less reliably identify them in other images.

In a particularly preferred embodiment, the present invention is used to achieve artificial contrast enhancement in a CT image that has been generated using an MRI contrast agent.

MRI contrast agents can in principle also be used in computed tomography; however, contrast enhancement is lower in computed tomography than in magnetic resonance imaging because the contrast of MRI contrast agents in MRI is based on a different physical effect than the contrast of MRI contrast agents in CT. Moreover, MRI contrast agents result in lower contrast enhancement in CT than customary CT contrast agents because they exhibit lower X-ray absorption. Nevertheless, it may be of interest to use an MRI contrast agent in CT, since there are MRI contrast agents which are specifically taken up by certain body cells or specifically bind to certain body cells, whereas there are no comparable CT contrast agents. Use of an MRI contrast agent in CT may thus visualize objects that were previously unidentifiable or not reliably identifiable in CT.

Available for magnetic resonance imaging are, for example, intracellular contrast agents which are specifically taken up by certain body cells. One example is the disodium salt of gadoxetic acid (Gd-EOB-DTPA disodium), which is described in U.S. Pat. No. 6,039,931A and is commercially available under the trade names Primovist® and Eovist®. This contrast agent is a so-called hepatobiliary contrast agent which is specifically taken up by liver cells (hepatocytes), accumulates in the functional tissue (parenchyma) and enhances contrast in healthy liver tissue. It is authorized for use in magnetic resonance imaging. There is no known comparable hepatobiliary contrast agent for computed tomography. This also applies to gadofosveset, an intravascular gadolinium-based MRI contrast agent which binds to blood serum albumin and thus leads to a long residence time of the contrast agent in the bloodstream (blood half-life about 17 hours). Another example of an intravascular contrast agent which is used in magnetic resonance imaging, but for which there is no comparable equivalent in computed tomography, is ferumoxytol, a colloidal iron-carbohydrate complex. Ferumoxytol may be given as an intravenous injection and is commercially available as a solution for intravenous injection under the trade names Rienso® and Ferahme®. The iron-carbohydrate complex has superparamagnetic properties and can therefore be used (off-label) for contrast enhancement in MRI examinations (see for example: L. P. Smits et al.: Evaluation of ultrasmall superparamagnetic iron-oxide (USPIO) enhanced MRI with ferumoxytol to quantify arterial wall inflammation, Atherosclerosis 2017, 263: 211-218).

A machine-learning model may be trained to learn the effect of increasing amounts of an MRI contrast agent on a representation of an examination region in a computed tomography examination.

The machine-learning model may be presented with representations of the examination region of a multiplicity of examination objects that represent the examination region in a CT examination after administration of different amounts of an MRI contrast agent. The maximum amount administered may be, for example, the standard amount of the MRI contrast agent as recommended or authorized for use thereof in MRI.

Once the machine-learning model is trained, what can be predicted are representations representing the examination region in a CT examination after administration of an amount of the MRI contrast agent greater than the standard amount.

The present invention can be performed wholly or partly with the aid of a computer system.

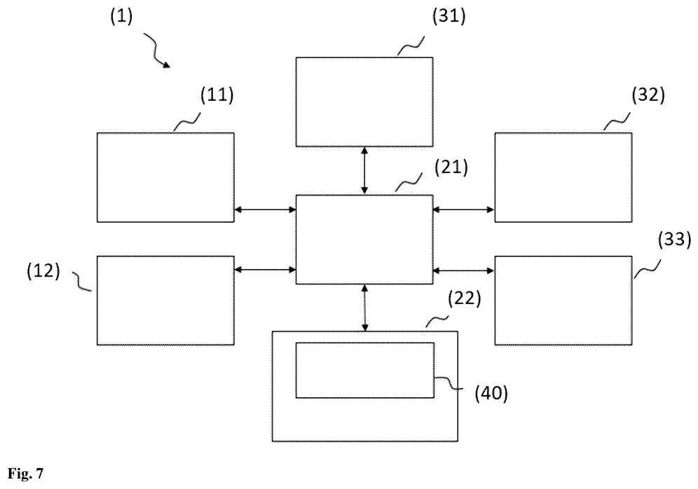

FIG. 7 shows by way of example and in schematic form such a computer system.

A “computer system” is an electronic data processing system that processes data by means of programmable calculation rules. Such a system typically comprises a “computer”, which is the unit that includes a processor for carrying out logic operations, and peripherals.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (1) shown in FIG. 7 comprises an input unit (10), a control and calculation unit (20) and an output unit (30).

The control and calculation unit (20) serves for control of the computer system (1), for coordination of the data flows between the units of the computer system (1), and for the performance of calculations.

The control and calculation unit (20) is configured
to cause the input unit (10) to receive a representation Rₚ of an examination region of an examination object, where the representation Rₚ represents the examination region without contrast agent or after administration of an amount aₚ of a contrast agent, where p is an integer less than n, where n is an integer greater than 2,
to feed the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region without contrast agent or after administration of a first amount a₁ of the contrast agent and each generated representation ^{T}Rⱼ* represents the examination region after an amount aⱼ of the contrast agent, where j is an index passing through integers from 2 to n, where the amounts a₁ to aₙ form a sequence of preferably increasing amounts, where the representation ^{T}R₂* is generated at least partly on the basis of the representation ^{T}R₁ and each further representation ^{T}Rⱼ* is generated at least partly on the basis of the respective previously generated representation ^{T}Rₖ₋₁* where k is an index passing through integers from 3 to n,
to receive from the machine-learning model one or more representations R_{p+q}* of the examination region, where each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1,
to cause the output unit (30) to store the one or more representations R_{p+q}* and/or to output them and/or to transmit them to a separate computer system.

FIG. 8 shows by way of example and in schematic form a further embodiment of the computer system according to the invention. The computer system (1) comprises a processing unit (21) connected to a memory (22). The processing unit (21) and the memory (22) form a control and calculation unit, as shown in FIG. 7.

The processing unit (21) may comprise one or more processors alone or in combination with one or more memories. The processing unit (21) may be customary computer hardware that is able to process information such as digital images (e.g., representation of the examination region), computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of same or of a different computer system.

The memory (22) may be customary computer hardware that is able to store information such as digital images (for example representations of the examination region), data, computer programs and/or other digital information either temporarily and/or permanently. The memory (22) may comprise a volatile and/or nonvolatile memory and may be nonremovable or removable. Examples of suitable memories are RAM (random access memory), ROM (read-only memory), a hard disk, a flash memory, an exchangeable computer floppy disk, an optical disc, a magnetic tape or a combination of the aforementioned. Optical discs can include compact discs with read-only memory (CD-ROM), compact discs with read/write function (CD-R/W), DVDs, Blu-ray discs and the like.

The processing unit (21) may be connected not just to the memory (22), but also to one or more interfaces (11, 12, 31, 32, 33) in order to display, transmit and/or receive information. The interfaces may comprise one or more communication interfaces (32, 33) and/or one or more user interfaces (11, 12, 31). The one or more communication interfaces (32, 33) may be configured to send and/or receive information, for example to and/or from an MRI scanner, a CT scanner, an ultrasound camera, other computer systems, networks, data memories or the like. The one or more communication interfaces (32, 33) may be configured to transmit and/or receive information via physical (wired) and/or wireless communication connections. The one or more communication interfaces (32, 33) may comprise one or more interfaces for connection to a network, for example using technologies such as mobile telephone, Wi-Fi, satellite, cable, DSL, optical fiber and/or the like. In some examples, the one or more communication interfaces (32, 33) may comprise one or more close-range communication interfaces configured to connect devices having close-range communication technologies such as NFC, RFID, Bluetooth, Bluetooth LE, ZigBee, infrared (e.g. IrDA) or the like.

The user interfaces (11, 12, 31) may comprise a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces (11, 12) are a microphone, an image- or video-recording device (for example a camera), a keyboard or a keypad, a joystick, a touch-sensitive surface (separate from a touchscreen or integrated therein) or the like. In some examples, the user interfaces (11, 12, 31) may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces (11, 12, 31) may furthermore comprise one or more interfaces for communication with peripherals such as printers and the like.

One or more computer programs (40) may be stored in the memory (22) and executed by the processing unit (21), which is thereby programmed to perform the functions described in this description. The retrieving, loading and execution of instructions of the computer program (40) may take place sequentially, such that an instruction is respectively retrieved, loaded and executed. However, the retrieving, loading and/or execution may also take place in parallel.

The machine-learning model according to the invention may also be stored in the memory (22).

The computer system of the present disclosure may be designed as a laptop, notebook, netbook and/or tablet PC; it may also be a component of an MRI scanner, a CT scanner or an ultrasound diagnostic device.

FIG. 9 shows schematically in the form of a flowchart an embodiment of the training method according to the invention. The training method (100) comprises the steps of:
(110) receiving training data,
   where the training data comprise representations ^{T}R₁ to ^{T}Rₙ of an examination region for a multiplicity of examination objects, where n is an integer greater than two,
   where each representation ^{T}Rᵢ represents the examination region after administration of an amount aᵢ of a contrast agent, where i is an integer in the range from 1 to n, where the amounts a₁ to aₙ form a sequence of preferably increasing amounts,
(120) training the machine-learning model,
   where the model is trained to generate, starting from the representation ^{T}R₁, representations ^{T}R₂* to ^{T}Rₙ* one after the other,
   where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n, where the representation ^{T}Rⱼ* represents the examination region after administration of the amount aₖ of the contrast agent and the representation ^{T}Rₖ₋₁* represents the examination region after administration of the amount aₖ₋₁ of the contrast agent,
   where differences between the generated representations ^{T}Rⱼ* and the representations ^{T}Rⱼ are quantified by means of a loss function, where j is an index passing through the numbers from 2 to n,
   where the differences are minimized in an optimization method by modifying model parameters of the machine-learning model,
(130) storing the trained machine-learning model and/or using the machine-learning model for prediction.

FIG. 10 shows schematically in the form of a flowchart an embodiment of the prediction method according to the invention. The prediction method (200) comprises the steps of
(210) receiving a representation Rₚ of the examination region,
   where the representation Rₚ represents the examination region without contrast agent or after administration of an amount aₚ of a contrast agent, where p is an integer less than n, where n is an integer greater than 2,
(220) feeding the representation Rₚ to a trained machine-learning model,
   where the machine-learning model has been trained on the basis of training data to generate, starting from a first representation ^{T}R₁, a number n−1 of representations ^{T}R₂* to ^{T}Rₙ* one after the other,
   where the first representation ^{T}R₁ represents the examination region without contrast agent or after administration of a first amount a₁ of the contrast agent and each generated representation ^{T}Rⱼ* represents the examination region after an amount aⱼ of the contrast agent, where j is an index passing through integers from 2 to n, where the amounts a₁ to aₙ form a sequence of preferably increasing amounts,
   where the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n,
(230) receiving one or more representations R_{p+q}* of the examination region from the machine-learning model,
   where each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1,
(240) outputting and/or storing and/or transmitting the one or more representations R_{p+q}*.

1. A computer-implemented method for training a machine-learning model, wherein the method comprises:
receiving training data;
wherein the training data comprise representations ^{T}R₁ to ^{T}Rₙ of an examination region for a multiplicity of examination objects, where n is an integer greater than two,
wherein each representation ^{T}Rᵢ represents the examination region after administration of an amount aᵢ of a contrast agent, where i is an integer in the range from 1 to n, where the amounts a₁ to aₙ form a sequence of preferably increasing amounts, and
wherein each examination object is preferably a human and the examination region is preferably part of the human;
training the machine-learning model;
wherein the model is trained to generate, starting from the representation ^{T}R₁, representations ^{T}R₂* to ^{T}Rₙ* one after the other,
wherein the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n, where the representation ^{T}Rₖ* represents the examination region after administration of the amount aₖ of the contrast agent and the representation ^{T}Rₖ₋₁* represents the examination region after administration of the amount aₖ₋₁ of the contrast agent,
wherein differences between the generated representations ^{T}Rⱼ* and the representations ^{T}Rⱼ are quantified by means of a loss function, where j is an index passing through the numbers from 2 to n, and
wherein the differences are minimized by modifying model parameters of the machine-learning model; and
storing the trained machine-learning model and/or using the machine-learning model for prediction.

2. The method of claim 1, further comprising-the-steps of:
for each generated representation ^{T}Rⱼ*: calculating a loss value for a pair composed of a representation ^{T}Rⱼ and the generated representation ^{T}Rⱼ* with the aid of a loss function, where j is an index passing through the numbers from 2 to n;
calculating a total loss value with the aid of a total loss function, where the total loss function is a function of the loss values; and
minimizing the total loss value by modifying the model parameters.

3. The method of claim 2, wherein the total loss function has the following formula:
$$LV = {\sum }_{j = 2}^{n} {w}_{j} \cdot {LF}_{j}$$
wherein LV is the total loss value, where LFⱼ are the loss functions for calculating the loss values for the differences between the representation ^{T}Rⱼ and the generated representation ^{T}Rⱼ* and wⱼ are weight factors, where j is an index passing through the numbers from 2 to n.

4. A computer-implemented method for predicting a representation of an examination region of an examination object, comprising:
receiving a representation Rₚ of the examination region;
wherein the representation Rₚ represents the examination region without contrast agent or after administration of an amount aₚ of a contrast agent, where p is an integer less than n, where n is an integer greater than 2, where the examination object is preferably a human and the examination region is preferably part of the human;
feeding the representation Rₚ to a trained machine-learning model;
wherein the machine-learning model has been trained on the basis of training data to generate, starting from a first representation ^{T}R₁, a number n−1 of representations ^{T}R₂* to ^{T}Rₙ* one after the other,
wherein the first representation ^{T}R₁ represents the examination region without contrast agent or after administration of a first amount a₁ of the contrast agent and each generated representation ^{T}Rⱼ* represents the examination region after an amount a₁ of the contrast agent, where j is an index passing through integers from 2 to n, where the amounts a₁ to aₙ form a sequence of preferably increasing amounts, and
wherein the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n;
receiving one or more representations R_{p+q}* of the examination region from the machine-learning model;
wherein each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1; and
outputting and/or storing and/or transmitting the one or more representations R_{p+q}*.

5. The method of claim 4, wherein the method comprises:
receiving the representation Rₚ of the examination region, where the representation Rₚ represents the examination region without contrast agent or after administration of the amount aₚ of the contrast agent, where p is an integer less than n,
feeding the representation Rₚ to the trained machine-learning model,
receiving the one or more representations R_{p+q}* of the examination region from the machine-learning model, where each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1, and
outputting and/or storing and/or transmitting the one or more representations R_{p+q}*.

6. The method according to claim 4, wherein each representation ^{T}R_{p+q}* and R_{p+q}* generated by the machine-learning model is returned to the trained machine-learning model in order to generate a representation ^{T}R_{p+q+1}* and R_{p+q+1}*.

7. The method according to claim 4, wherein m is in the range from n to n+10.

8. The method according to claim 1, wherein each generated representation is calculated according to the following formula:
$${M}^{q} ( {R}_{1} ) = {R}_{1 + q}^{*}$$
where M represents a transformation which is applied by the machine-learning model to input data of the machine-learning model, where M^{q} means the q-times application of the transformation, where in a first step the transformation is applied to a first representation R₁, in a second step the transformation is applied to the result of the application in the first step in that the result is passed back into the machine-learning model and the procedure is repeated with each further result of a transformation until the transformation has been applied a total of q times, where q is an integer which may assume the values of 1 to m, where m is an integer less than n−1 or equal to n−1 or greater than n−1.

9. The method according to claim 4, wherein the examination region is the human liver or is part of the human liver.

10. The method according to claim 4, wherein the received representation Rₚ is a CT image, MRI image or ultrasound image.

11. The method of claim 4, wherein the received representation Rₚ is a CT image and wherein the contrast agent is an MRI contrast agent.

12. (canceled)

13. A computer program product comprising a computer program that can be loaded into a working memory of a computer system, where it causes the computer system to execute the following:
receiving a representation Rₚ of an examination region of an examination object, where the representation Rₚ represents the examination region without contrast agent or after administration of an amount aₚ of a contrast agent, where p is an integer less than n, where n is an integer greater than 2, where the examination object is preferably a human and the examination region is preferably part of the human,
feeding the representation Rₚ to a trained machine-learning model, where the machine-learning model has been trained on the basis of training data to generate, starting from a first representation ^{T}R₁, a number n−1 of representations ^{T}R₂* to ^{T}Rₙ* one after the other, where the first representation ^{T}R₁ represents the examination region without contrast agent or after administration of a first amount a₁ of the contrast agent and each generated representation ^{T}Rⱼ* represents the examination region after an amount a₁ of the contrast agent, where j is an index passing through integers from 2 to n, where the amounts a₁ to aₙ form a sequence of preferably increasing amounts, where the representation ^{T}R₂* is generated at least partly on the basis of the representation ^{T}R₁ and each further representation ^{T}Rⱼ* is generated at least partly on the basis of the respective previously generated representation ^{T}Rₖ₋₁*, where k is an index passing through integers from 3 to n,
receiving one or more representations R_{p+q}* of the examination region from the machine-learning model, where each of the one or more representations R_{p+q}* represents the examination region after administration of the amount a_{p+q} of the contrast agent, where q is an index passing through the numbers from 1 to m, where m is an integer less than n−1 or equal to n−1 or greater than n−1, and
outputting and/or storing and/or transmitting the one or more representations R_{p+q}*.

14-15. (canceled)

16. A kit comprising a contrast agent and ti computer program product of claim 13.

17. The method of claim 4, where the trained machine-learning model has been trained, according to a computer-implemented method comprising:
receiving training data:
wherein the training data comprise representations ^{T}R₁ to ^{T}Rₙ of an examination region for a multiplicity of examination objects, where n is an integer greater than two,
wherein each representation ^{T}Rⱼ represents the examination region after administration of an amount aⱼ of a contrast agent, where i is an integer in the range from 1 to n, where the amounts a₁ to aₙ form a sequence of preferably increasing amounts, and
wherein each examination object is preferably a human and the examination region is preferably part of the human;
training the machine-learning model:
wherein the model is trained to generate, starting from the representation ^{T}R₁, representations ^{T}R₂* to ^{T}Rₙ* one after the other,
wherein the representation ^{T}R₂* is generated at least partly on the basis of the representation ^{T}R₁ and each further representation ^{T}Rₖ* is generated at least partly on the basis of the respective previously generated representation ^{T}Rₖ₋₁*, where k is an index passing through integers from 3 to n, where the representation ^{T}Rₖ* represents the examination region after administration of the amount aₖ of the contrast agent and the representation ^{T}Rₖ₋₁* represents the examination region after administration of the amount aₖ₋₁ of the contrast agent,
wherein differences between the generated representations ^{T}Rⱼ* and the representations ^{T}Rⱼ are quantified by means of a loss function, where j is an index passing through the numbers from 2 to n, and
wherein the differences are minimized by modifying model parameters of the machine-learning model; and
storing the trained machine-learning model and/or using the machine-learning model for prediction.

18. The method of claim 1, wherein each representation ^{T}R_{p+q}* generated by the machine-learning model is returned to the machine-learning model in order to generate a representation ^{T}R_{p+q+1}*.

19. The method according to claim 1, wherein each generated representation is calculated according to the following formula:
$${M}^{q} ( {}^{T} {R}_{1} ) = {}^{T} {R}_{1 + q}^{*}$$
where M represents a transformation which is applied by the machine-learning model to input data of the machine-learning model, where M^{q} means the q-times application of the transformation, where in a first step the transformation is applied to a first representation ^{T}R₁, in a second step the transformation is applied to the result of the application in the first step in that the result is passed back into the machine-learning model and the procedure is repeated with each further result of a transformation until the transformation has been applied a total of q times, where q is an integer which may assume the values of 1 to m, where m is an integer less than n−1 or equal to n−1 or greater than n−1.

20. The method of claim 1, wherein the examination region is the human liver or is part of the human liver.

21. The method of claim 1, wherein the received representations are CT images, MRI images or ultrasound images.

22. The method of claim 1, wherein the received representations are CT images and wherein the contrast agent is an MRI contrast agent.
